(19) United States
(12) Patent Application Publication
(10) Pub. No.: US 2024/0150857 A1
(43) Pub. Date: May 9, 2024

(54) METHOD AND DEVICE FOR POWER ALLOCATION IN CARBON DIOXIDE INCUBATOR, AND CARBON DIOXIDE INCUBATOR

(71) Applicants: QINGDAO HAIER BIOMEDICAL TECHNOLOGY CO., LTD., Qingdao (CN); QINGDAO HAIER BIOMEDICAL CO., LTD., Qingdao (CN)

(72) Inventors: Huan CHEN, Qingdao (CN); Zhanjie LIU, Qingdao (CN); Haitao CHEN, Qingdao (CN); Xianshuang TANG, Qingdao (CN); Wei HU, Qingdao (CN); Yuantong XIA, Qingdao (CN); Zepeng DUAN, Qingdao (CN); Huanwen JU, Qingdao (CN)

(73) Assignees: QINGDAO HAIER BIOMEDICAL TECHNOLOGY CO., LTD., Qingdao (CN); QINGDAO HAIER BIOMEDICAL CO., LTD., Qingdao (CN)

(21) Appl. No.: 18/550,441

(22) PCT Filed: Jun. 21, 2022
(86) PCT No.: PCT/CN2022/100136

§ 371 Date: Sep. 13, 2023

(30) Foreign Application Priority Data
Jul. 21, 2021 (CN) 202110825839.9

(51) Int. Cl. C12Q 3/00; C12M 1/00

(52) U.S. Cl. CPC C12Q 3/00; C12M 41/14

(57) ABSTRACT

Disclosed are a method and a device for power allocation in a carbon dioxide incubator, and a carbon dioxide incubator. The method for power allocation in a carbon dioxide incubator provided with heating wires on multiple inner surfaces of the carbon dioxide incubator includes: according to a current temperature and a target temperature of a box body of the carbon dioxide incubator, determining a power index; in case where the current temperature is less than or equal to the target temperature, according to the power index, determining a heating strategy for each inner surface of the carbon dioxide incubator by looking up a table; and according to the heating strategy for each inner surface, adjusting at least one or more of the power and/or the start-stop time of the heating wires on each inner surface.

### CROSS-REFERENCE OF RELATED APPLICATIONS

The present application is a national stage of the International Application PCT/CN2022/100136, filed on Jun. 21, 2022, which claims priority to Chinese Patent Application No. CN202110825839.9, filed on Jul. 21, 2021, the contents of which is incorporated herein by reference in their entirety.

### TECHNICAL FIELD

The present disclosure relates to the technical field of temperature control, for example, relates to a method and a device for power allocation in a carbon dioxide incubator, and a carbon dioxide incubator.

### BACKGROUND

The carbon dioxide incubator is a device for culturing cells/tissues in vitro by simulating a similar environment inside an organism for the growth of the cells/tissues in the incubator. It is an advanced instrument for culturing cells, tissue, and bacteria. It is the key equipment necessary for immunology, oncology, genetics, and bioengineering. This kind of equipment has high requirements for temperature, humidity, and internal environment. However, the condensation problem often occurs during the use of carbon dioxide incubators, and condensation water will breed bacteria, which is not allowed in carbon dioxide incubators.

In the related arts, there is a technical solution to solve the condensation problem by controlling the power of multiple heating wires on each surface of the carbon dioxide incubator. Usually, the power of multiple heating wires on each surface is controlled by using multiple sets of PID (Proportion, Integration, Differentiation) algorithms to control the power of each heating wire separately or by controlling the power of multiple heating wires on each surface through thyristors on one path.

### SUMMARY

In order to have a basic understanding of some aspects of the disclosed embodiments, a brief summary is given below. The summary is not intended to be a general comment or to identify crucial/essential constituent elements or to describe the protection scope of these embodiments but rather to serve as a preface to the detailed description that follows.

The embodiment of the present disclosure provides a method and a device for power allocation in a carbon dioxide incubator, and a carbon dioxide incubator, in order to improve the efficiency of controlling the power of heating wires on each surface of the carbon dioxide incubator and save system resources.

In some embodiments, the carbon dioxide incubator is provided with heating wires on multiple inner surfaces thereof, and the method comprises:
according to a current temperature and a target temperature of a box body of the carbon dioxide incubator, determining a power index;
in a case where the current temperature is less than or equal to the target temperature, according to the power index, determining a heating strategy for each inner surface of the carbon dioxide incubator by looking up a table; and
according to the heating strategy for each inner surface, adjusting the power and/or the start-stop time of the heating wires on each inner surface.

In some embodiments, the device comprises a processor and a memory storing program instructions, wherein the processor is configured to execute the mentioned method for a carbon dioxide incubator when executing the program instructions.

In some embodiments, the carbon dioxide incubator comprises the mentioned device for a carbon dioxide incubator.

The method and the device for power allocation in a carbon dioxide incubator, and a carbon dioxide incubator provided by the embodiment of the disclosure can have the following technical advantages:

The power index is obtained according to the current temperature and the target temperature; the heating strategies for each inner surface of the carbon dioxide incubator are obtained by the power index, and the power and/or the start-stop time of the heating wires on each inner surface is/are adjusted based on the heating strategy; thus achieving separate control of the heating wires on the inner surfaces of the carbon dioxide incubator for preventing condensation in the carbon dioxide incubator. There is no need to control the heating wires by multiple sets of control algorithms, which reduces algorithm redundancy, improves control efficiency, and saves system resources.

The above general description and the description below are exemplary and explanatory only and are not intended to limit the present disclosure.

### BRIEF DESCRIPTION OF THE DRAWINGS

One or more embodiments are illustrated by means of the corresponding drawings, which do not constitute a limitation of the embodiments, elements having the same reference numerals in the drawings are shown as similar elements, and the drawings do not constitute a limitation of proportion, wherein:

FIG. 1 is a schematic diagram of a method for power allocation in a carbon dioxide incubator provided by an embodiment of the present disclosure;

FIG. 2 is a schematic diagram of a method for power allocation in a carbon dioxide incubator provided by the other embodiment of the present disclosure;

FIG. 3 is a schematic diagram of a method for power allocation in a carbon dioxide incubator provided by the other embodiment of the present disclosure;

FIG. 4 is a schematic diagram of a method for power allocation in a carbon dioxide incubator provided by an embodiment of the present disclosure; and

FIG. 5 is a schematic diagram of a device for power allocation in a carbon dioxide incubator provided by an embodiment of the present disclosure.

### DETAILED DESCRIPTION OF THE EMBODIMENTS

In order to enable a more detailed understanding of the features and technical content of the embodiments of the present disclosure, the implementation of the embodiments of the present disclosure will be described in detail below in conjunction with the accompanying drawings, which are for illustration only and are not intended to limit the embodiments of the present disclosure. In the following technical description for convenience of explanation, several details are provided for a full understanding of the disclosed embodiments. However, one or more embodiments may still be practiced without these details. In other instances, the well-known structures and devices may simplify the disclosure in order to simplify the drawings.

The terms “first”, “second” and the like in the specification and claims of embodiments of the present disclosure and the above drawings are used to distinguish similar elements and are not necessarily used to describe a particular order or priority. It should be understood that the data used in this way can be interchanged where appropriate for the embodiments of the present disclosure described herein. Furthermore, the terms “include” and “have” and any variations thereof are intended to cover a non-exclusive inclusion.

Unless otherwise illustrated, the term “a plurality of” means two or more.

In the embodiment of the present disclosure, the character “I” indicates that the front element and rear element are in an “or” relationship. For example, A/B illustrates A or B.

The term “and/or” is an association relationship that describes elements, indicating that there can be three relationships. For example, “A and/or B” represents relationships: A or B, or A and B.

The term “corresponding to” can refer to an association or binding relationship, while A corresponding to B refers to an association or binding relationship between A and B.

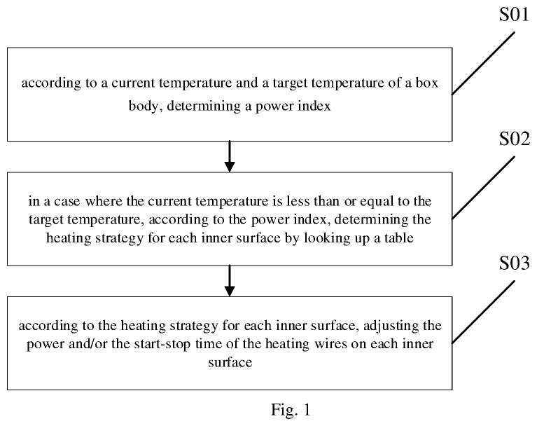

With reference to FIG. 1, a method for power allocation in a carbon dioxide incubator provided by the embodiment of the present disclosure, comprises:
S01, according to a current temperature and a target temperature of a box body, determining a power index;
S02, in a case where the current temperature is less than or equal to the target temperature, according to the power index, determining the heating strategy for each inner surface by looking up a table; and
S03, according to the heating strategy for each inner surface, adjusting the power and/or the start-stop time of the heating wires on each inner surface.

With regard to the method for power allocation in the carbon dioxide incubator provided by the embodiment of the present disclosure, the power index is obtained based on the current temperature and the target temperature; the heating strategies for each inner surface of the carbon dioxide incubator are obtained by the power index, and the power and/or the start-stop time of the heating wires on each inner surface is/are adjusted based on the heating strategy; thus achieving separate control of the heating wires on each inner surface of the carbon dioxide incubator for preventing condensation in the carbon dioxide incubator. In the present embodiment, one power index can be obtained by one set of algorithms. The heating strategy of the heating wires on each inner surface of the carbon dioxide incubator can be obtained by looking up the table with the power index. There is no need to control the heating wires by multiple sets of control algorithms, which reduces algorithm redundancy, improves control efficiency, and saves system resources.

Optionally, the step of, according to the current temperature and the target temperature of the box body, determining the power index comprises: obtaining the current temperature; inputting the current temperature and the target temperature into a PID algorithm; and outputting the power index.

In this way, using the PID algorithm, the power index can be obtained by taking the current temperature and the target temperature inside the carbon dioxide incubator as input values for the PID algorithm. Compared to the related arts that employ multiple sets of PID algorithms to calculate the heating power of each inner surface of the carbon dioxide incubator separately, the power index is calculated by using one set of PID algorithms in this embodiment which reduces control redundancy caused by multiple sets of PID algorithms, saves system resources, and improves control efficiency.

Optionally, the step of, according to the power index, determining the heating strategy for each inner surface of the carbon dioxide incubator by looking up the table(s) comprises: according to a pre-set power table, determining a power of the heating wires on each inner surface corresponding to the power index; and/or according to a pre-set time table, determining an output period of the heating wires on each inner surface corresponding to the power index.

In this way, based on the pre-set power table, the power of the heating wire on each inner surface corresponding to the power table is looked up with the power index, that is, the control strategy of the heating wires on each inner surface can be obtained by one power index. Or, based on the pre-set time table, the output period of the heating wires on each inner surface can be obtained at the corresponding position on the time table with one power index. Or, based on the pre-set power table and the pre-set time table, the power and the output period of the heating wires on each inner surface can be obtained with one power index.

Optionally, the step of, according to the heating strategy for each inner surface, adjusting the power and/or the start-stop time of the heating wires on each inner surface comprises: according to the power of the heating wires on each inner surface, adjusting the power of the heating wires on each inner surface to the set power; and/or according to the output period of the heating wires on each inner surface, controlling the start-stop time of the heating wires on each inner surface.

The start-stop time of the heating wires on each inner surface of the carbon dioxide incubator can be controlled by a thyristor. Specifically, the thyristor can be provided on multiple inner surfaces of the carbon dioxide incubator, with one thyristor corresponding to the heating wire(s) on one inner surface. The thyristor is assigned a value based on the determined output period of heating wires on each inner surface to control the start and stop of the heating wires.

In this way, the power of the heating wires on each inner surface is controlled to the set power according to the power of the heating wires on each inner surface determined by the power table, to achieve separate control of the power of the heating wires on each inner surface of the carbon dioxide incubator, reasonably allocate the power of the heating wires on each inner surface, and prevent condensation. Or, the start-stop time of the heating wires on each inner surface of the carbon dioxide incubator is controlled by the output period of the heating wires on each inner surface determined by the time table, and the output period of the heating wires in a heating stage is controlled by controlling the start and stop of the heating wires on each inner surface, thus, the power of the heating wires is allocated, separate control of multiple inner surfaces of the carbon dioxide incubator is achieved, and condensation is prevented. Or, the power and the start-stop time of the heating wires on each inner surface are controlled and the power and the output period of the heating wires on each inner surface are determined by the power table and the time table, thus, separating control of multiple inner surfaces of the carbon dioxide incubator is achieved, and condensation is prevented.

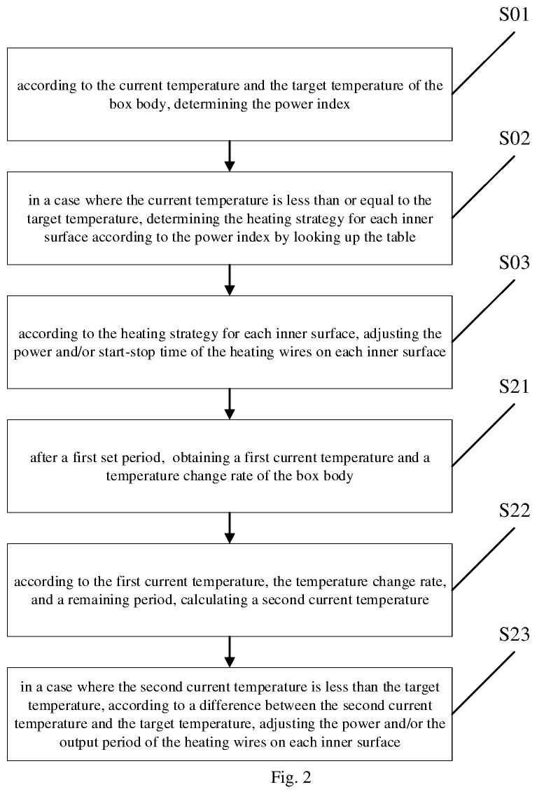

With reference to FIG. 2, the method for power allocation in the carbon dioxide incubator provided by the embodiment of the present disclosure comprises:
S01, according to the current temperature and the target temperature of the box body, determining the power index;
S02, in a case where the current temperature is less than or equal to the target temperature, determining the heating strategy for each inner surface according to the power index by looking up the table;
S03, according to the heating strategy for each inner surface, adjusting the power and/or start-stop time of the heating wires on each inner surface.
S21, after a first set period, obtaining a first current temperature and a temperature change rate of the box body;
S22, according to the first current temperature, the temperature change rate, and a remaining period, calculating a second current temperature; and
S23, in a case where the second current temperature is less than the target temperature, according to a difference between the second current temperature and the target temperature, adjusting the power and/or the output period of the heating wires on each inner surface;
where the remaining period is a difference between a set period to reach the target temperature and the first set period.

With regard to the method for power allocation in the carbon dioxide incubator provided by the embodiment of the present disclosure, after the remaining period whether the temperature inside the carbon dioxide incubator will reach the target temperature can be predicted by obtaining the first current temperature and the temperature change rate of the box body of the carbon dioxide incubator. If the target temperature is not reached, it indicates that the heating strategy of each inner surface of the carbon dioxide incubator cannot meet the demand at this time, and condensation may occur. Therefore, the heating strategy is redefined based on the difference between the predicted second current temperature and the target temperature, and the power and/or the output period of the heating wires on each inner surface is/are adjusted according to the redefined heating strategy. This avoids unexpected situations in the carbon dioxide incubator after determining the heating strategy and adjusting the heating wires according to the heating strategy, makes it impossible to achieve the target temperature within the set period, and improves the applicability of the carbon dioxide incubator.

Optionally, the step of according to the first current temperature and the temperature change rate, calculating the second current temperature comprises: calculating T2=T1+t×V; where T2 is the second current temperature, T1 is the first current temperature, t is the remaining period, and V is the temperature change rate.

In this way, the temperature change value after the remaining period is obtained by calculating the product of the remaining period and the temperature change rate, and the second current temperature after the remaining period is predicted by summing the temperature change value and the current detected first current temperature.

Optionally, the step of, according to the difference between the second current temperature and the target temperature, adjusting the power of the heating wires on each inner surface comprises: according to a pre-set correspondence, determining a power of the heating wires on each inner surface corresponding to the difference; and according to the target power of the heating wires, adjusting the power of the heating wires on each inner surface to the corresponding power.

In this way, the target power of the heating wires on each inner surface corresponding to the difference is determined according to the pre-set correspondence, in order to determine control strategies for the heating wires on each inner surface of the carbon dioxide incubator. And according to the target power of the heating wires, the power of the heating wires on each inner surface is adjusted to the corresponding power. The heating wires on each inner surface are controlled according to the corresponding control strategy, so that the carbon dioxide incubator can reach the target temperature within the set duration, in order to achieve a response to unexpected situations and improve the applicability of the carbon dioxide incubator.

Optionally, the step of according to the difference between the second current temperature and the target temperature, adjusting the output period of the heating wires on each inner surface comprises: according to the pre-set correspondence, determining the output period of the heating wires on each inner surface corresponding to the difference; and according to the output period of the heating wires, controlling the start-stop time of the heating wires on each inner surface.

In this way, the output period on each inner surface corresponding to the difference is determined according to the pre-set correspondence, in order to determine control strategies for the heating wires on each inner surface of the carbon dioxide incubator. And the start-stop time of the heating wires on each inner surface is controlled according to the output period of the heating wires. The heating wires on each inner surface are controlled according to the corresponding control strategy, so that the carbon dioxide incubator can reach the target temperature within the set duration, in order to achieve a response to unexpected situations and improve the applicability of the carbon dioxide incubator.

Optionally, the step of according to the difference between the second current temperature and the target temperature, adjusting the power and the output period of the heating wires on each inner surface comprises: according to the pre-set correspondence, determining the target power and the output period of the heating wires on each inner surface corresponding to the difference; according to the target power of the heating wires, adjusting the power of the heating wires on each inner surface to the corresponding power; and according to the output period of the heating wires, controlling the start-stop time of the heating wires on each inner surface.

In this way, the target power and the output period of the heating wires on each inner surface corresponding to the difference are determined according to the pre-set correspondence, in order to determine control strategies for the heating wires on each inner surface of the carbon dioxide incubator. Further, according to the target power of the heating wires, the power of the heating wires on each inner surface is adjusted to the corresponding power. According to the output period of the heating wires, the start-stop time of the heating wires on each inner surface is controlled. The heating wires on each inner surface are controlled according to the corresponding control strategy, so that the carbon dioxide incubator can reach the target temperature within the set duration, in order to achieve a response to unexpected situations and improve the applicability of the carbon dioxide incubator.

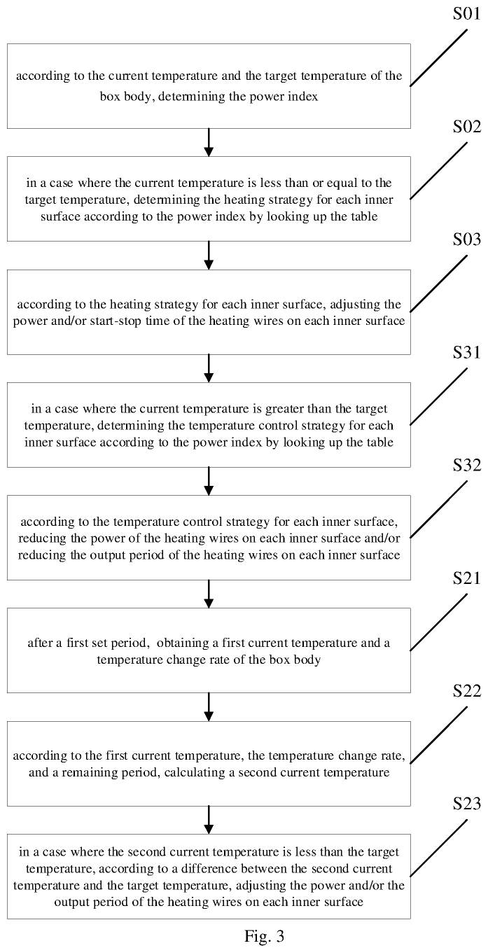

With reference to FIG. 3, the method for power allocation in the carbon dioxide incubator provided by the embodiment of the present disclosure comprises:

S01, according to the current temperature and the target temperature of the box body, determining the power index;
S02, in a case where the current temperature is less than or equal to the target temperature, according to the power index, determining the heating strategy for each inner surface according to the power index by looking up the table;
S03, according to the heating strategy for each inner surface, adjusting the power and/or the start-stop time of the heating wires on each inner surface;
S31, in a case where the current temperature is greater than the target temperature, determining the temperature control strategy for each inner surface according to the power index by looking up the table;
S32, according to the temperature control strategy for each inner surface, reducing the power of the heating wires on each inner surface and/or reducing the output period of the heating wires on each inner surface;
S21, after the first set period, obtaining the first current temperature and the temperature change rate of the box body;
S22, according to the first current temperature, the temperature change rate, and the remaining period, calculating a second current temperature; and
S23, in a case where the second current temperature is less than the target temperature, according to the difference between the second current temperature and the target temperature, adjusting the power and/or the output period of the heating wires on each inner surface;
where the remaining period is the difference between the set period to reach the target temperature and the first set period.

With regard to the method for power allocation in the carbon dioxide incubator provided by the embodiment of the present disclosure, in a case where the current temperature inside the carbon dioxide incubator is greater than the target temperature, the heating wires on each inner surface of the carbon dioxide incubator are controlled again at this time to blindly improve heating efficiency, that will damage the preservation of the samples inside the carbon dioxide incubator. Therefore, it is necessary to determine corresponding control strategies for the carbon dioxide incubator. In a case where the current temperature is greater than the target temperature, the temperature control strategy for each inner surface of the carbon dioxide incubator is determined according to the power index by looking up the table, and the power of the heating wires on each inner surface is reduced according to the temperature control strategy for each inner surface, or the output period of the heating wires on each inner surface is reduced according to the temperature control strategy for each inner surface, or the power and the output period of the heating wires on each inner surface are reduced according to the temperature control strategy for each inner surface. This can reduce the heat output of the heating wires on each inner surface and reduce heating efficiency, so as to ensure the biological preservation function inside the carbon dioxide incubator to avoid damage to internal samples.

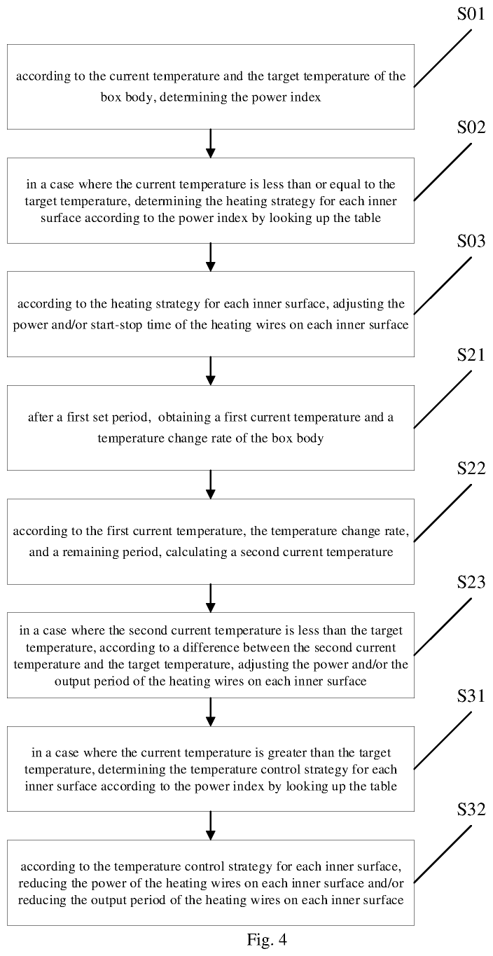

With reference to FIG. 4, the method for power allocation in the carbon dioxide incubator provided by the embodiment of the present disclosure comprises:
S01, according to the current temperature and the target temperature of the box body, determining the power index;
S02, in a case where the current temperature is less than or equal to the target temperature, according to the power index, determining the heating strategy for each inner surface according to the power index by looking up the table;
S03, according to the heating strategy for each inner surface, adjusting the power and/or the start-stop time of the heating wires on each inner surface;
S21, after the first set period, obtaining the first current temperature and the temperature change rate of the box body;
S22, according to the first current temperature, the temperature change rate, and the remaining period, calculating the second current temperature; and
S23, in a case where the second current temperature is less than the target temperature, according to a difference between the second current temperature and the target temperature, adjusting the power and/or the output period of the heating wires on each inner surface;
S31, in a case where the current temperature is greater than the target temperature, determining the temperature control strategy for each inner surface according to the power index by looking up the table;
S32, according to the temperature control strategy for each inner surface, reducing the power of the heating wires on each inner surface and/or reducing the output period of the heating wires on each inner surface;
where the remaining period is the difference between the set period to reach the target temperature and the first set period.

With regard to the method for power allocation in the carbon dioxide incubator provided by the embodiment of the present disclosure, in a case where the current temperature inside the carbon dioxide incubator is greater than the target temperature, the heating wires on each inner surface of the carbon dioxide incubator are controlled again at this time to blindly improve heating efficiency, that will damage the preservation of the samples inside the carbon dioxide incubator. Therefore, it is necessary to determine corresponding control strategies for the carbon dioxide incubator. In a case where the current temperature is greater than the target temperature, the temperature control strategy for each inner surface of the carbon dioxide incubator is determined according to the power index by looking up the table, and according to the temperature control strategy for each inner surface, the power of the heating wires on each inner surface is reduced, or the output period of the heating wires on each inner surface is reduced, or the power and the output period of the heating wires on each inner surface are reduced. This can reduce the heat output of the heating wires on each inner surface and reduce heating efficiency, so as to ensure the biological preservation function inside the carbon dioxide incubator to avoid damage to internal samples.

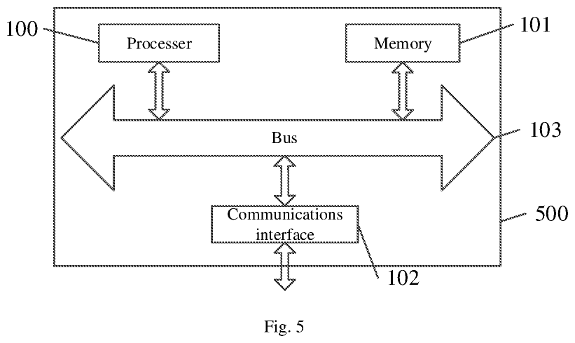

With reference to FIG. 5, the embodiment of the present disclosure provides a device 500 for power allocation in a carbon dioxide incubator, which comprises a processor 100 and a memory 101. Optionally, the device 500 may also include a communication interface 102 and a bus 103. The processor 100, the communication interface 102, and the memory 101 are communicated with each other through the bus 103. The communication interface 102 may be configured for information transmission. The processor 100 may invoke logic instructions in the memory 101 to execute the method for power allocation in a carbon dioxide incubator in the above-mentioned embodiment.

Further, the logic instructions in the memory 101 described above may be realized in the form of software functional units and may be stored in a computer-readable storage medium when sold or used as a separate product.

As a computer-readable storage medium, the memory 101 may be configured to store software programs, and computer executable programs, such as program instructions/modules corresponding to the methods in embodiments of the present disclosure. The processor 100 executes the function application and data processing by running the program instructions/modules stored in the memory 101 that is to implement the method for power allocation in a carbon dioxide incubator in the above-mentioned embodiment.

The memory 101 may include a stored program area and a stored data area wherein the stored program area may store an operating system and an application program required for at least one function. The storage data area may store data created according to the use of the terminal device. In addition, the memory 101 may include a high-speed random access memory and may also include a non-volatile memory.

The embodiment of the present disclosure provides a carbon dioxide incubator, in which multiple inner surfaces are equipped with heating wires and the heating wires on each inner surface being controlled by the corresponding thyristor, comprising: the above-mentioned device for power allocation in a carbon dioxide incubator.

The embodiments of the present disclosure provide a computer-readable storage medium storing computer-executable instructions configured to execute the above-mentioned method for power allocation in a carbon dioxide incubator.

The computer-readable storage medium may be a transient computer-readable storage medium or a non-transient computer-readable storage medium.

The technical solution of the embodiment of the present disclosure can be embodied in the form of a software product, The computer software product is stored in a storage medium and includes one or more instructions for causing a computer device (which may be a personal computer, server, or network device, etc.) to perform all or part of the steps of the method described in the embodiment of the present disclosure. The above mentioned storage medium may be a non-transient storage medium, including a USB disk, a mobile hard disk, a Read-Only Memory (ROM), a Random Access Memory (RAM), a magnetic disk or an optical disk, and other media capable of storing program codes or maybe a transient storage medium.

The above description and drawings sufficiently illustrate embodiments of the present disclosure to enable practice by those skilled in the art. Other embodiments may include structural logical electrical procedural and other modifications. The embodiments represent only possible variations. Unless explicitly required, individual parts and functions are optional, and the order of operation can vary. Portions and features of some embodiments may be included in or in place of portions and features of other embodiments. Furthermore, the terms used in the present disclosure are used only to describe embodiments and are not used to limit the claims. As used in the embodiments and the description of the claims, the singular forms of “a”, “an” and “the” are intended to include the plural forms as well, unless the context clearly indicates. Similarly, the term “and/or” as used in this application means encompassing one or more associated lists of any and all possible combinations. Additionally, when used in this application, the term “comprise” and its variants “comprises” and/or “comprising”, etc. refer to the presence of stated features, totals, steps, operations, elements, and/or components, but do not exclude the presence or addition of one or more other features, totals, steps, operations, elements, components, and/or groupings of these. In the absence of further limitations, an element defined by the phrase “comprises a/an . . . ” does not preclude the existence of another identical element in the process, method, or device in which the element is included. Herein each embodiment may be highlighted as being different from the other embodiments and the same similar parts between the various embodiments may be referred to with respect to each other. For the method, product, etc. disclosed by the embodiment, if it corresponds to the method portion disclosed by the embodiment, reference can be made to the description of the method portion where relevant.

Those skilled in the art will appreciate that the various example units and algorithm steps described in connection with the embodiments disclosed herein can be implemented in electronic hardware or a combination of computer software and electronic hardware. Whether these functions are performed in hardware or software can depend on the specific application and design constraints of the technical solution. The skilled artisan may use different methods for each particular application to implement the described functionality but such implementation should not be considered outside the scope of the disclosed embodiments. It will be apparent to the skilled person that for convenience and conciseness of description, the specific operating processes of the above-mentioned systems, devices, and units may be referred to the corresponding processes in the mentioned method embodiments and will not be repeated herein.

In the embodiments disclosed herein, the disclosed methods, and products (including but not limited to devices, devices, etc.) may be implemented in other ways. For example, the above-mentioned embodiment of the device is only schematic, for example, the division of the unit may be only a logical function division, and in practice, there may be another division mode, for example, multiple units or components may be combined or integrated into another system, or some features may be ignored or not performed. In addition, the coupling or direct coupling or communication connection between each other shown or discussed may be indirect coupling or communication connection through some interface, device, or unit, and may be electrical, mechanical, or another form. The elements illustrated as separate elements may or may not be physically separated, and the elements displayed as elements may or may not be physical elements, i.e. may be located in one place or may be distributed over a plurality of network elements. Some or all of the units can be selected according to actual needs to realize the embodiment. In addition, each functional unit in the embodiment of the present disclosure may be integrated into one processing unit, each unit may exist physically alone, or two or more units may be integrated into one unit.

The flowcharts and block diagrams in the accompanying drawings illustrate the architecture functionality and operation of possible implementations of systems methods and computer program products according to embodiments of the present disclosure. In this regard, each block in a flow chart or block diagram may represent a module, program segment, or part of code containing one or more executable instructions for performing a specified logical function. In some alternative implementations, the functions indicated in the boxes may also occur in a different order than those indicated in the drawings. For example, two successive boxes can actually be executed substantially in parallel, or they can sometimes be executed in reverse order, depending on the functionality involved. In the description corresponding to the flowcharts and block diagrams in the drawings, the operations or steps corresponding to different blocks may also occur in a different order than that disclosed in the description, and sometimes there is no specific order between the different operations or steps. For example, two successive operations or steps can actually be performed substantially in parallel, or they can sometimes be performed in reverse order, depending on the functionality involved. Each block in the block diagram and/or flow chart, and a combination of the blocks in the block diagram and/or flow chart, may be implemented in a dedicated hardware-based system that performs a specified function or action or may be implemented in a combination of dedicated hardware and computer instructions.

1. A method for power allocation in a carbon dioxide incubator provided with heating wires on multiple inner surfaces of the carbon dioxide incubator, comprising:
according to a current temperature and a target temperature of a box body of the carbon dioxide incubator, determining a power index;
in case where the current temperature is less than or equal to the target temperature, according to the power index, determining a heating strategy for each inner surface of the carbon dioxide incubator by looking up a table; and
according to the heating strategy for each inner surface, adjusting at least one or more of the power and the start-stop time of the heating wires on each inner surface.

2. The method according to claim 1, wherein the according to the current temperature and the target temperature of the box body of the carbon dioxide incubator, determining the power index comprises:
obtaining the current temperature; and
inputting the current temperature and the target temperature into a PID algorithm and outputting the power index.

3. The method according to claim 1, wherein the according to the power index, determining the heating strategy for each inner surface of the carbon dioxide incubator by looking up the table comprises:
according to a pre-set power table, determining the power of the heating wires on each inner surface corresponding to the power index.

4. The method according to claim 3, wherein the according to the heating strategy for each inner surface, adjusting at least one or more of the power and the start-stop time of the heating wires on each inner surface comprises:
according to the power of the heating wires on each inner surface, adjusting the power of the heating wires on each inner surface to the set power.

5. The method according to claim 1, after according to the heating strategy for each inner surface, adjusting at least one or more of the power and the start-stop time of the heating wires on each inner surface, further comprising:
after a first set period, obtaining a first current temperature and a temperature change rate of the box body of the carbon dioxide incubator;
according to the first current temperature, the temperature change rate, and a remaining period, calculating a second current temperature;
in case where the second current temperature is less than a target temperature, according to a difference between the second current temperature and the target temperature, adjusting the power of the heating wires on each inner surface;
wherein the remaining period is a difference between a set period to reach the target temperature and the first set period;
wherein the according to the first current temperature and the temperature change rate, calculating the second current temperature comprises:
calculating T2=T1+t×V;
wherein T2 is the second current temperature, T1 is the first current temperature, t is the remaining period, and V is the temperature change rate.

6. (canceled)

7. The method according to claim 5, wherein the according to the difference between the second current temperature and the target temperature, adjusting the power of the heating wires on each inner surface comprises:
according to a pre-set correspondence, determining a target power of the heating wires on each inner surface corresponding to the difference; and
according to the target power of the heating wires, adjusting the power of the heating wires on each inner surface to the corresponding power.

8. The method according to claim 1, after according to the current temperature and the target temperature of the box body of the carbon dioxide incubator, determining the power index, further comprising:
in case where the current temperature is greater than the target temperature, determining the temperature control strategy for each inner surface of the carbon dioxide incubator according to the power index by looking up the table; and
according to the temperature control strategy for each inner surface, reducing at least one or more of the power and the output period of the heating wires on each inner surface.

9. A device for power allocation in a carbon dioxide incubator, comprising a processor and a memory storing program instructions, wherein
the processor is configured to execute the method for power allocation in a carbon dioxide incubator according to claim 1 when executing the program instructions.

10. (canceled)

11. The method according to claim 1, wherein the according to the power index, determining the heating strategy for each inner surface of the carbon dioxide incubator by looking up the table comprises:
according to a pre-set time table, determining an output period of the heating wires on each inner surface corresponding to the power index.

12. The method according to claim 11, wherein the according to the heating strategy for each inner surface, adjusting at least one or more of the power and the start-stop time of the heating wires on each inner surface comprises:
according to the output period of the heating wires on each inner surface, controlling the start-stop time of the heating wires on each inner surface.

13. The method according to claim 1, after according to the heating strategy for each inner surface, adjusting at least one or more of the power and the start-stop time of the heating wires on each inner surface, further comprising:
after a first set period, obtaining a first current temperature and a temperature change rate of the box body of the carbon dioxide incubator;
according to the first current temperature, the temperature change rate, and a remaining period, calculating a second current temperature;
in case where the second current temperature is less than the target temperature, according to a difference between the second current temperature and the target temperature, adjusting the output period of the heating wires on each inner surface;
wherein the remaining period is a difference between a set period to reach the target temperature and the first set period;
wherein the according to the first current temperature and the temperature change rate, calculating the second current temperature comprises:
calculating T2=T1+t×V;
wherein T2 is the second current temperature, T1 is the first current temperature, t is the remaining period, and V is the temperature change rate.

14. The method according to claim 13, wherein the according to the difference between the second current temperature and the target temperature, adjusting the output period of the heating wires on each inner surface comprises:
according to a pre-set correspondence, determining the output period of the heating wires on each inner surface corresponding to the difference; and
according to the output period of the heating wires, controlling the start-stop time of the heating wires on each inner surface.

15. The method according to claim 3, after according to the heating strategy for each inner surface, adjusting at least one or more of the power and the start-stop time of the heating wires on each inner surface, further comprising:
after a first set period, obtaining a first current temperature and a temperature change rate of the box body of the carbon dioxide incubator;
according to the first current temperature, the temperature change rate, and a remaining period, calculating a second current temperature;
in case where the second current temperature is less than the target temperature, according to a difference between the second current temperature and the target temperature, adjusting the power of the heating wires on each inner surface;
wherein the remaining period is a difference between a set period to reach the target temperature and the first set period;
wherein the according to the first current temperature and the temperature change rate, calculating the second current temperature comprises:
calculating T2=T1+t×V;
wherein T2 is the second current temperature, T1 is the first current temperature, t is the remaining period, and V is the temperature change rate.

16. The method according to claim 4, after according to the heating strategy for each inner surface, adjusting at least one or more of the power and the start-stop time of the heating wires on each inner surface, further comprising:
after a first set period, obtaining a first current temperature and a temperature change rate of the box body of the carbon dioxide incubator;
according to the first current temperature, the temperature change rate, and a remaining period, calculating a second current temperature;
in case where the second current temperature is less than the target temperature, according to a difference between the second current temperature and the target temperature, adjusting the power of the heating wires on each inner surface;
wherein the remaining period is a difference between a set period to reach the target temperature and the first set period;
wherein the according to the first current temperature and the temperature change rate, calculating the second current temperature comprises:
calculating T2=T1+t×V;
wherein T2 is the second current temperature, T1 is the first current temperature, t is the remaining period, and V is the temperature change rate.

17. The method according to claim 11, after according to the heating strategy for each inner surface, adjusting at least one or more of the power and the start-stop time of the heating wires on each inner surface, further comprising:
after a first set period, obtaining a first current temperature and a temperature change rate of the box body of the carbon dioxide incubator;
according to the first current temperature, the temperature change rate, and a remaining period, calculating a second current temperature;
in case where the second current temperature is less than the target temperature, according to a difference between the second current temperature and the target temperature, adjusting the output period of the heating wires on each inner surface;
wherein the remaining period is a difference between a set period to reach the target temperature and the first set period;
wherein the according to the first current temperature and the temperature change rate, calculating the second current temperature comprises:
calculating T2=T1+t×V;
wherein T2 is the second current temperature, T1 is the first current temperature, t is the remaining period, and V is the temperature change rate.

18. The method according to claim 12, after according to the heating strategy for each inner surface, adjusting at least one or more of the power and the start-stop time of the heating wires on each inner surface, further comprising:
after a first set period, obtaining a first current temperature and a temperature change rate of the box body of the carbon dioxide incubator;
according to the first current temperature, the temperature change rate, and a remaining period, calculating a second current temperature;
in case where the second current temperature is less than the target temperature, according to a difference between the second current temperature and the target temperature, adjusting the output period of the heating wires on each inner surface;
wherein the remaining period is a difference between a set period to reach the target temperature and the first set period;
wherein the according to the first current temperature and the temperature change rate, calculating the second current temperature comprises:
calculating T2=T1+t×V;
wherein T2 is the second current temperature, T1 is the first current temperature, t is the remaining period, and V is the temperature change rate.

19. The method according to claim 3, after according to the current temperature and the target temperature of the box body of the carbon dioxide incubator, determining the power index, further comprising:
in case where the current temperature is greater than the target temperature, determining the temperature control strategy for each inner surface of the carbon dioxide incubator according to the power index by looking up the table; and
according to the temperature control strategy for each inner surface, reducing the power of the heating wires on each inner surface.

20. The method according to claim 11, after according to the current temperature and the target temperature of the box body of the carbon dioxide incubator, determining the power index, further comprising:
in case where the current temperature is greater than the target temperature, determining the temperature control strategy for each inner surface of the carbon dioxide incubator according to the power index by looking up the table; and
according to the temperature control strategy for each inner surface, reducing the output period of the heating wires on each inner surface.

21. The method according to claim 5, after according to the current temperature and the target temperature of the box body of the carbon dioxide incubator, determining the power index, further comprising:
in case where the current temperature is greater than the target temperature, determining the temperature control strategy for each inner surface of the carbon dioxide incubator according to the power index by looking up the table; and
according to the temperature control strategy for each inner surface, reducing the power of the heating wires on each inner surface.

22. The method according to claim 13, after according to the current temperature and the target temperature of the box body of the carbon dioxide incubator, determining the power index, further comprising:
in case where the current temperature is greater than the target temperature, determining the temperature control strategy for each inner surface of the carbon dioxide incubator according to the power index by looking up the table; and
according to the temperature control strategy for each inner surface, reducing the output period of the heating wires on each inner surface.
